(19) United States
(12) Patent Application Publication
(10) Pub. No.: US 2012/0264827 A1
(43) Pub. Date: Oct. 18, 2012

(54) SULFANYLAMIDE DERIVATIVES, USES THEREOF AND COMPOSITIONS COMPRISING THEM

(75) Inventors: Meir Bialer, Jerusalem (IL); Boris Yagen, Jerusalem (IL); Hen Naama, Jerusalem (IL)

(73) Assignee: YISSUM RESEARCH DEVELOPMENT COMPANY OF THE HEBREW UNIVERSITY OF JERUSALEM, LTD., Givat Ram, Jerusalem (IL)

(21) Appl. No.: 13/497,434

(22) PCT Filed: Sep. 21, 2010
(86) PCT No.: PCT/IL2010/000776

§ 371 Date: Mar. 21, 2012

Related U.S. Application Data
(60) Provisional application No. 61/244,097, filed on Sep. 21, 2009.

(51) Int. Cl. A61K 31/18; A61P 25/08; C07C 311/46

(52) U.S. Cl. 514/603; 564/86

(57) ABSTRACT

The present invention concerns a family of sulfanilamide derivatives of formula (I) as anticonvulsant agents, where R is selected from optionally substituted C₄-C₉ alkyl, optionally substituted C₆-C₁₀ aryl, optionally substituted C₆-C₁₀ alkylenearyl and optionally substituted C₅-C₁₀heteroaryl; R₂ is selected from —H and optionally substituted C₁-C₆ alkyl; each of R₃ and R₄, independently of each other, is selected from —H, optionally substituted C₁-C₆ alkyl, optionally substituted C₆-C₁₀ aryl and optionally substituted C₅-C₁₀ heteroaryl; n is 0, 1, 2, 3 or 4. The derivatives have been prepared and their anticonvulsant profile was evaluated for the control of epileptic seizures.

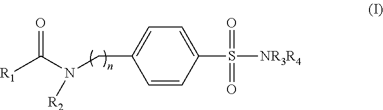

### FIELD OF THE INVENTION

This invention relates to sulfanilamide derivatives with improved anticonvulsant potency and low toxicity.

### BACKGROUND OF THE INVENTION

Epilepsy is a chronic disorder of the brain characterized by an enduring predisposition to generate epileptic seizures, and by the neurobiological, cognitive, psychological and associated social consequences. In spite of the large therapeutic arsenal of old and new antiepileptic drugs (AEDs), about 30% of epileptic patients are not seizure-free. In many cases the clinical use of AEDs is restricted by their side effects, thus requiring the development of new chemical entities to be at least as effective as existing AEDs, but demonstrating diminished side effects.

Valproic acid (VPA; 2-propylpentanoic acid) is a branched monocarboxylic acid with eight carbon atoms and optimal chemical structure in regard to efficacy and safety margin between anticonvulsant activity and sedative/hypnotic adverse effects. Many analogues and derivatives of VPA were synthesized in an attempt to find superior compounds that would retain the anticonvulsant activity which is correlated with the basic structure of VPA and at the same time would not cause the adverse effects associated with VPA's use.

Amidation of VPA leads to the formation of valpromide (VPD), a compound with improved anticonvulsant potency and lower teratogenicity in animal models. However, these advantages in animals do not have clinical implications, since in humans VPD serves as a prodrug of VPA. Amide derivatives of VPA's constitutional isomers were found to be metabolically stable and significantly more potent as anticonvulsants compared to their corresponding acids.

Derivatives of 4-amino-benzenesulfonamide (sulfanilamide) in MES and scMet seizure tests have been found to display excellent anticonvulsant activity profile [1]. Acetazolamide is an old AED, containing a sulfonamide group in its structures. Various aromatic sulfonamide derivatives of VPA demonstrated the influence of different sulfonamide moieties on their anticonvulsant activity [2, 3, 4, 5]. It was shown that 5-valproylamido-1,3,4-thiamidazole-2-sulfonamide, an heterocyclic sulfonamide, displayed strong anticonvulsant activity in the MES test in mice [2]. The analogue, 5-(2,2,3,3-tetramethylcyclopropanecarboxamide)-1,3,4-thiamidazole-2-sulfonamide also demonstrated potent anticonvulsant activity and a protective index (PI=TD₅₀/ED₅₀) above 50 in MES test in rats [3]. Tasso et al. have reported that 4-(valproylamido)-benzenesulfonamide is a potent anticonvulsant in MES test in mice [5].

2,2,3,3-tetramethylcyclopropanecarboxylic acid (TMCA is a cyclopropyl analogue of VPA displaying weak anticonvulsant activity and small safety margin. Recently, it was found that 4-(2,2,3,3-tetramethylcyclopropanecarboxamido)-benezenesulfonamide (TMCD-benzenesulfonamide was potent in a mouse MES test and exhibited high potency and lack of toxicity in a rat MES test [4].

### REFERENCES

[1] Ganz, A. J.; Waser, P. G.; Pfirrmann, R. W. [Development of new antiepileptics. V. Pharmacological activity of some derivatives of sulfanilamide Arzneimittelforschung 1978, 28, 1331-1334.
[2] Masereel, B.; Rolin, S.; Abbate, F.; Scozzafava, A.; Supuran, C. T. Carbonic anhydrase inhibitors: anticonvulsant sulfonamides incorporating valproyl and other lipophilic moieties. J. Med. Chem. 2002, 45, 312-320.
[3] Okada, A.; Onishi, Y.; Yagen, B.; Shimshoni, J. A.; Kaufmann, D. et al. Tetramethylcyclopropyl analogue of the leading antiepileptic drug, valproic acid: evaluation of the teratogenic effects of its amide derivatives in NMRI mice. Birth Defects Res. A Clin. Mol. Teratol. 2008, 82, 610-621.
[4] Shimshoni, J. A.; Bialer, M.; Yagen, B. Synthesis and anticonvulsant activity of aromatic tetramethylcyclopropanecarboxamide derivatives. Bioorg. Med. Chem. 2008, 16, 6297-6305.
[5] Tasso, S. M.; Moon, S.; Bruno-Blanch, L. E.; Estiu, G. L. Characterization of the anticonvulsant profile of valpromide derivatives. Bioorg. Med. Chem. 2004, 12, 3857-3869.

### SUMMARY OF THE INVENTION

Since several of the currently available antiepileptic drugs (AEDs) have been associated with severe side effects and also have been failing in the control of seizures in about 30% of epileptic patients, there has been a substantial need for the development of new, more effective and less toxic AEDs.

The inventors of the present invention have thus further explored the design of sulfanilamide derivatives, particularly those having branched aliphatic carboxylic acids and phenylacetic acid as new and improved anticonvulsant agents. As will be demonstrated hereinbelow, the novel derivatives have been prepared and their anticonvulsant profile was evaluated in two of the most widely used animal models of epilepsy: rat maximal electroshock seizure test (MES) and the subcutaneous pentylenetetrazole seizure test (scMet) models.

In a first aspect, the present invention provides a compound of the general formula (I):

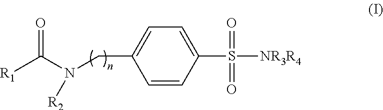

wherein

R₁ is selected from optionally substituted C₄-C₉ alkyl, optionally substituted C₆-C₁₀ aryl, optionally substituted C₆-C₁₀ alkylenearyl and optionally substituted C₅-C₁₀-heteroaryl;

R₂ is selected from —H and optionally substituted C₁-C₆ alkyl;

each of R₃ and R₄, independently of each other, is selected from —H, optionally substituted C₁-C₆ alkyl, optionally substituted C₆-C₁₀ aryl and optionally substituted C₅-C₁₀-heteroaryl;

n is0, 1, 2, 3 or 4;

excluding the compound wherein R₁ is t-butyl and each of R₂, R₃ and R₄ is —H and excluding the compound wherein R₁ is benzyl and each of R₂, R₃ and R₄ is —H.

Within the scope of the present invention, the term “alkyl” refers to a carbon chain, linear or branched, optionally substituted with one or more substituent as defined herein. The designation “—C₄-C₉-alkyl” refers to an alkyl, as defined, having between 4 and 8 carbon atoms, which may be linear or branched. Non-limiting examples of such alkyl group are iso-butyl, n-butyl, sec-butyl, tert-butyl, n-pentyl, 2-pentyl, 3-pentyl, n-hexyl, 2-hexyl, 3-hexyl, n-heptyl, 1-iso-propylbutyl, 2-iso-propylbutyl, 3-heptyl, 4-(3-methyl)hexyl, 2,2-dimethyl-pent-3-yl, 2,4-dimethyl-pent-3-yl, 2,2,4-trimethyl-pent-3-yl, 2,2-dimethyl-prop-1-yl, 2-methylhex-3-yl, 4-methylhex-3-yl, 2,4-dimethyl-hex-3-yl, 3-methyl-hept-4-yl, 2,2-dimethyl-hex-3-yl, 5-methyl-hex-3-yl, 2-methyl-but-1-yl, and so on.

Similarly, the designation “C₁-C₆ alkyl” refers to an alkyl, as defined, having between 1 and 6 carbon atoms, which may be linear or branched. Non-limiting examples of such alkyl group, apart from those already mentioned above, are methyl, ethyl, propyl, iso-propyl, iso-butyl, n-butyl, sec-butyl, tert-butyl, n-pentyl, 2-pentyl, 3-pentyl, n-hexyl, 2-hexyl, 3-hexyl, 1-iso-propylbutyl, 2-iso-propylbutyl, 2,2-dimethyl-pent-3-yl, 2,4-dimethyl-pent-3-yl, 2,2,4-trimethyl-pent-3-yl, 2,2-dimethyl-prop-1-yl, 2-methylhex-3-yl, 4-methylhex-3-yl, 2,4-dimethyl-hex-3-yl, 3-methyl-hept-4-yl, 2,2-dimethyl-hex-3-yl, 5-methyl-hex-3-yl, 2-methyl-but-1-yl, and others.

The term “alkylenearyl” refers to an alkyl carbon chain having repeating —CH₂— groups, which is substituted at one end with an aryl group as defined and on the other end being substituted to the C═O group of the compounds of formula (I). Similarly, the term “—C₆-C₁₀-alkylearyl” refers to an alkylenearyl, as defined, having between 6 and 10 carbon atoms, including the —CH₂— groups and the aryl carbon atoms. The alkylene portion may be linear or branched. Non-limiting examples of such alkylenearyl are methylenearyl (—CH₂—Ar), ethylenearyl (—CH₂—CH₂—Ar), propylenearyl (—CH₂CH₂CH₂—Ar), iso-propylenearyl (—CH(CH₃)CH₂—Ar) and n-butylenearyl (—CH₂CH₂CH₂CH₂—Ar); the Ar group in some embodiments is phenyl.

The term “—C₆-C₁₀-aryl” refers to an aromatic monocyclic or multicyclic group containing from 6 to 10 carbon atoms. Aryl groups include, but are not limited to groups such as unsubstituted or substituted fluorenyl, unsubstituted or substituted phenyl, and unsubstituted or substituted naphthyl.

In some embodiments of the invention, the aryl is a phenyl ring. Where the C₆-C₁₀ aryl or the aryl group of the C₆-C₁₀ alkyleneary is substituted, the substitution on the aryl group, e.g., a phenyl ring, may be by one, two, three, four or five atoms or groups. Mono-substitution refers to the replacement of a single hydrogen atom of the phenyl ring with a different atom or group of atoms. The hydrogen to be replaced may be one of the ortho-hydrogens (positions 2 or 6), meta-hydrogens (positions 3 or 5) or para-hydrogen (position 4) to the existing exo-cyclic bond. For example, substitution of the phenyl ring by two atoms or groups may be at both ortho-positions (positions 2 and 6), at one ortho-position and one meta-position (positions 2 and 3 or positions 2 and 5), at one ortho-position and at the para-position (positions 2 and 4), at both meta-positions (positions 3 and 5), or at one meta-position and at one para-position (positions 3 and 4).

Similarly, tri-substitution of a phenyl ring may be at positions 2,3,4 or 2,3,5 or 2,3,6 or 2,4,6 or 2,4,5 or 3,4,5.

As used herein, “—C₅-C₁₀-heteroaryl” refers to a monocyclic or multicyclic aromatic ring system having between 5 and 10 carbon atoms and at least one heteroatom selected from N, O and S in the ring system. The heteroaryl group may be optionally fused to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl and isoquinolinyl,

The term “optionally substituted” refers, in its broadest definition, to the replacement of a hydrogen atom present, in a group which is said of being optionally substituted, by another atom such as a halide (Br, Cl, I and F) or a group comprising a heteroatom such as S, O, and/or N, the groups being for example selected from —OH, —NH₂, —NO₂, etc.

The radical group NR₃R₄ is an amine which may be —NH₂ (where each of R₃ and R₄ is —H), a primary amine of the form —NHR₄ (wherein R₃ is —H and R₄ is selected as defined) or a tertiary amine of the form —NR₃R₄, wherein each of R₃ and R₄ is selected as defined herein.

In some embodiments, the —NR₃R₄ may be further protonated or alkylated to provide a quaternary ammonium group (positively charged) associated with an appropriate negatively charged atom or group, e,g., halide (charged Cl, Br, I or F), hydroxide, etc.

In some embodiments, in the compound of formula (I), R₁ is a C₄-C₉ alkyl. In other embodiments, said C₄-C₉ alkyl is selected from iso-butyl, n-butyl, sec-butyl, tert-butyl, n-pentyl, 3-pentyl, n-hexyl, 2-hexyl, 3-hexyl, n-heptane, n-hexane, 1-iso-propylbutyl, 2-iso-propylbutyl, 2,2-dimethyl-pent-3-yl, 2,4-dimethyl-pent-3-yl, 2,2,4-trimethyl-pent-3-yl, 2,2-dimethyl-prop-1-yl, 2-methylhex-3-yl, 4-methylhex-3-yl, 2,4-dimethyl-hex-3-yl, 3-methyl-hept-4-yl, 2,2-dimethyl-hex-3-yl, 5-methyl-hex-3-yl, and 2-methyl-but-1-yl.

In some embodiments, R₁ is selected from 3-pentyl and 2-iso-propylbutyl. In other embodiments, R₁ is 3-pentyl and the compound of the invention is a compound of general formula (II):

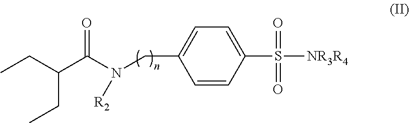

wherein each of n, R₂, R₃ and R₄ is as defined hereinabove.

In some embodiments, in a compound of formulae (I) and (II), R₂ is —H.

In other embodiments, in a compound of formula (I) and (II), each of R₃ and R₄ is —H.

In other embodiments, in a compound of formulae (I) and (II), each of R₂, R₃ and R₄ is H.

In further embodiments, in a compound of formulae (I) and (II), n is zero, 1 or 2.

In other embodiments, R₁ is 2-iso-propylbutyl and the compound of the invention is a compound of general formula (III):

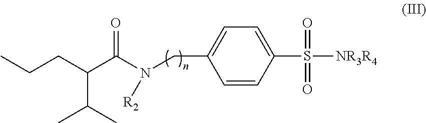

wherein each of n, R₂, R₃ and R₄ is as defined hereinabove.

In some embodiments, in a compound of formula (III), R₂ is —H.

In other embodiments, in a compound of formula (III), each of R₃ and R₄ is —H.

In other embodiments, in a compound of formula (III), each of R₂, R₃ and R₄ is —H.

In other embodiments of the invention, the compounds of formula (I) are selected amongst compounds of formula (IV):

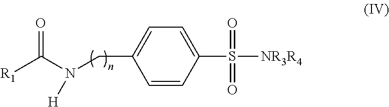

wherein each of n, R₁, R₃ and R₄ is as defined hereinabove.

In further embodiments, the compounds of the invention are selected amongst compounds of the general formula (V):

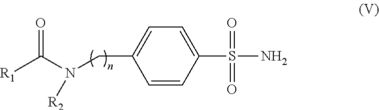

wherein each of n, R₁ and R₂ is as defined hereinabove.

The invention further provides the novel compounds listed in Table 1 below, excluding compounds designated therein Compound 9 and Compound 30.

In some embodiments, the compounds of the invention are compounds designated in Table 1 as Compound 10, 11, 12, 13, 14, 15, 16, 17, 18, 19, 20, 21, 22, 23, 24, 25, 26, 27, 28 and 29. In other embodiments, the compounds of the invention are Compounds designated in Table 1 as Compound 10 and/or 11 and/or 12 and/or 13 and/or 14 and/or 15 and/or 16 and/or 17 and/or 18 and/or 19 and/or 20 and/or 21 and/or 22 and/or 23 and/or 24 and/or 25 and/or 26 and/or 27 and/or 28 and/or 29. In other embodiments, the compounds of the invention are compounds designated in Table 1 as Compound 10 or 11 or 12 or 13 or 14 or 15 or 16 or 17 or 18 or 19 or 20 or 21 or 22 or 23 or 24 or 25 or 26 or 27 or 28 or 29.

It is to be understood that the compounds of formulae (I)-(V) may contain chiral centers. Such chiral centers may be of either the (R) or (S) configuration, or may be a mixture thereof. Thus, the compounds provided herein may be enantiomerically pure, or be stereoisomeric or diastereomeric mixtures. It is also to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

In another aspect of the present invention, there is provided the use of a compound of the general formula (I), as defined above, for the preparation of a composition. In some embodiments, said composition is a pharmaceutical composition.

In still another aspect of the present invention, there is provided the use of a compound of formula (I):

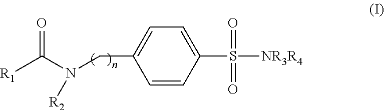

in the preparation of a pharmaceutical composition for the treatment and/or prevention of a disease or disorder, wherein:

R₁ is selected from optionally substituted C₄-C₉ alkyl, optionally substituted C₆-C₁₀ aryl, optionally substituted C₆-C₁₀ alkylenearyl and optionally substituted C₅-C₁₀-heteroaryl;

R₂ is selected from —H and optionally substituted C₁-C₆ alkyl;

n is 0, 1, 2, 3 or 4; and

each of R₃ and R₄, independently of each other, is selected from —H, optionally substituted C₁-C₆ alkyl, optionally substituted C₆-C₁₀ aryl and optionally substituted C₅-C₁₀-heteroaryl.

In some embodiments, in the compound of formula (I) used in the preparation of a pharmaceutical composition, R₁ is a C₄-C₉ alkyl or a C₆-C₁₀ alkylenearyl.

In some embodiments, a compound of formula (I) for use as defined is a compound wherein R₂ is —H.

In other embodiments, in a compound of formula (I) for use as defined is a compound wherein each of R₃ and R₄ is —H.

In other embodiments, in a compound of formula (I) for use as defined is a compound wherein each of R₂, R₃ and R₄ is —H.

In other embodiments, said C₄-C₉ alkyl is selected from iso-butyl, n-butyl, sec-butyl, tert-butyl, n-pentyl, 3-pentyl, n-hexyl, 2-hexyl, 3-hexyl, n-heptane, n-hexane, 1-iso-propylbutyl, 2-iso-propylbutyl, 2,2-dimethyl-pent-3-yl, 2,4-dimethyl-pent-3-yl, 2,2,4-trimethyl-pent-3-yl, 2,2-dimethyl-prop-1-yl, 2-methylhex-3-yl, 4-methylhex-3-yl, 2,4-dimethyl-hex-3-yl, 3-methyl-hept-4-yl, 2,2-dimethyl-hex-3-yl, 5-methyl-hex-3-yl, and 2-methyl-but-1-yl.

In some embodiments, R₁ is selected from tert-butyl, 3-pentyl and 2-iso-propylbutyl.

In some embodiments, the compounds used in the preparation of said compositions are at least one of compounds herein designated Compound 1 and Compound 2.

In further embodiments, R₁ is tert-butyl and the compound of general formula (I) is a compound of formula (VI):

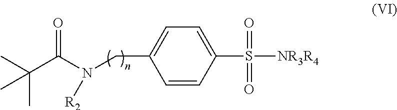

wherein each of n, R₂, R₃ and R₄ is as defined hereinabove.

In further embodiments, R₁ is a C₆-C₁₀ alkylenearyl, being selected in a non-limiting fashion from —CH₂—Ar, —CH₂—CH₂—Ar and —CH₂CH₂CH₂—Ar. In some embodiments, the aryl group (—Ar) is phenyl and the alkylenearyl is benzyl. The compound of the invention is, thus, a compound of the formula (VII):

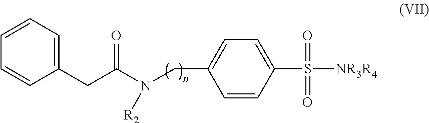

wherein each of n, R₂, R₃ and R₄ is as defined hereinabove.

In some embodiments, in a compound of formulae (VI) and (VII), R₂ is —H.

In other embodiments, in a compound of formula (VI) and (VII), each of R₃ and R₄ is —H.

In other embodiments, in a compound of formulae (VI) and (VII), each of R₂, R₃ and R₄ is —H.

In some embodiments, the compound to be used in the preparation of a pharmaceutical composition, as defined, or in the treatment of a disease or disorder, as defined, is any one compound of Table 1, herein designated Compounds 9-30.

In some embodiments, the compounds for use as defined are selected from the compounds designated in Table 1 as Compound 9, 10, 11, 12, 13, 14, 15, 16, 17, 18, 19, 20, 21, 22, 23, 24, 25, 26, 27, 28, 29 and 30.

In other embodiments, the compounds are selected from any one of Compound 9, 11, 16, 18, 23, 25, 28, 29 and 30.

In some embodiments, the composition is for use in the treatment or prevention of epilepsy.

In another aspect of the present invention, there is provided a use of any one compound of the invention in the treatment or prevention of epilepsy.

The invention further provides in another of its aspects, a pharmaceutical composition comprising at least one compound according to formula (I). In some embodiments, said composition is for use in the treatment and/or prevention of epilepsy.

In some embodiments, said pharmaceutical composition comprising also a pharmaceutically acceptable diluent, carrier or excipient.

In some further embodiments, the pharmaceutical composition comprises at least one compound selected from Compounds 9-30 of Table 1.

In some embodiments, the compounds are selected from any one of Compound 9, 11, 16, 18, 23, 25, 28, 29 and 30.

The pharmaceutically acceptable carriers employed herein, for example, vehicles, adjuvants, excipients, or diluents, are well-known to those who are skilled in the art and are readily available to the public. It is preferred that the pharmaceutically acceptable carrier be one which is chemically inert to the active compounds and one which has no detrimental side effects or toxicity under the conditions of use.

The choice of carrier will be determined in part by the particular active agent, as well as by the particular method used to administer the composition. Accordingly, there is a wide variety of suitable formulations of the pharmaceutical composition of the present invention. The following formulations for oral, aerosol, parenteral, subcutaneous, intravenous, intramuscular, interperitoneal, rectal, and vaginal administration are merely exemplary and are in no way limiting.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia, as well as pastilles comprising the active ingredient in an inert base, such as gelatin and glycerin, or sucrose and acacia, emulsions, gels, and the like containing, in addition to the active ingredient, such carriers as are known in the art.

The compounds of the present invention, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. The compounds can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as polyethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxy-ethylenepolypropylene copolymers, (d) amphoteric detergents such as, for example, alkyl-β-aminopriopionates, and 2-alkyl-imidazoline quaternary ammonium salts, and (3) mixtures thereof.

The parenteral formulations will typically contain from about 0.5 to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The compounds of the present invention may be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4^{th} ed., pages 622-630 (1986).

Additionally, the compounds of the present invention may be made into suppositories by mixing with a variety of bases, such as emulsifying bases or water-soluble bases. Formulations suitable for vaginal administration may be presented as tampons, creams, gels, pastes, foams, or spray formulas containing, in addition to the active ingredient, such carriers as are known in the art to be appropriate

In another aspect of the invention, there is provided a method for the treatment and/or prevention of a disease or disorder, comprising administering to a subject in need thereof an effective amount of a composition comprising at least one compound of the formula (I):

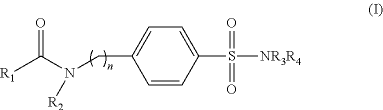

wherein

R₁ is selected from optionally substituted C₄-C₉ alkyl, optionally substituted C₆-C₁₀ aryl, optionally substituted C₆-C₁₀ alkylenearyl and optionally substituted C₅-C₁₀-heteroaryl;

R₂ is selected from —H and optionally substituted C₁-C₆ alkyl;

each of R₃ and R₄, independently of each other, is selected from —H, optionally substituted C₁-C₆ alkyl, optionally substituted C₆-C₁₀ aryl and optionally substituted C₅-C₁₀-heteroaryl; and

n is 0, 1, 2, 3, or 4.

In some embodiments, said disease or disorder is epilepsy.

In another aspect of the present invention, there is provided a method for the treatment and/or prevention of epilepsy, said method comprising administering an effective amount of a composition of at least one compound of the formula (I), as defined.

The “effective amount” of a compound according to the present invention, or a composition comprising thereof according to the invention, used in for purposes herein, is determined by such considerations as may be known in the art. The amount must be effective to achieve the desired therapeutic effect as described above, i.e. treatment and/or prevention of epilepsy depending, inter alia, on the type and severity of the disease to and the existing treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile within the body, a variety of pharmacological parameters such as half-life in the body, on undesired side effects, if any, on factors such as age and gender, etc.

The term “treatment and/or prevention” or any lingual variation thereof, as used herein refers to the administering of a therapeutic amount of the composition of the present invention which is effective to ameliorate undesired symptoms associated with the disease, to prevent the manifestation of such symptoms before they occur, to slow down the progression of the disease, slow down the deterioration of symptoms, to enhance the onset of remission period, slow down the irreversible damage which may be caused by the disease, to delay epileptic attacks, to lessen the severity or cure the disease, to improve more rapid recovery, or to prevent the disease form occurring or a combination of two or more of the above.

### DETAILED DESCRIPTION OF EMBODIMENTS

The inventors of the present invention have developed a new class of anticonvulsant aromatic amides which may be obtained by the coupling of phenylacetic acid or branched aliphatic carboxylic acids with 4-amino-benzenesulfonamide or derivatives thereof.

As will be further detailed below, eight compounds of the compounds of the invention have demonstrated unique anticonvulsant activity with rat-MES-ED₅₀ values of less than 50 mg/kg (Table 3). The ED₅₀ values of the most active sulfonamides 11 and 16 was about 10 mg/kg (Table 3) and was comparable to that of the new AEDs. 2-Ethyl-N-(4-sulfamoyl-phenyl)-butyramide (Compound 11, Table 1) was found the most active compound with an anticonvulsant potency 49 times greater than that of valproic acid, VPA. The compound has only six carbon atoms in its carboxylic moiety, as opposed to eight carbons of VPA. The rat-MES PI value of Compound 11, of >50, was determined to be about 31 times higher than that of VPA. A homologue of Compound 11, 2-methyl-N-(4-sulfamoyl-phenyl)-pentanamide, Compound 16, showed similar anticonvulsant properties as Compound 11 (Table 3).

The anticonvulsant properties of the compounds of the invention make them candidates as new potent and safe AEDs.

A large variety of amide derivatives of VPA and its analogues have been synthesized and assayed as anticonvulsants. Previous studies have shown that 4-(valproylamido)-benzenesulfonamide (6) was potent as an anticonvulsant in the mice-MES test (ED₅₀=21 mg/kg). The 4-(2,2,3,3-tetramethylcyclopropanecarboxamido)-benzenesulfon amide (7), a cyclic analogue of 6 previously synthesized by the inventors, showed similar high anticonvulsant potency in the MES test in mice and rats (ED₅₀=26 mg/kg). This indicates that benzenesulfonamide derivatives of VPA and TMCA showed better anticonvulsant potency and higher protective index than VPA.

A series of aromatic sulfonamides containing 4-amino-benzenesulfonamide moiety coupled with branched aliphatic carboxylic acid with 5-9 carbon atoms or with phenylacetic acid in their lipophilic moieties have been synthesized and their anticonvulsant activity and neurotoxicity was evaluated. Table 1 and 2 present the anticonvulsant activity and the neurotoxicity of Compound 9-30 in mice and rats, respectively. Among the synthesized derivatives containing five carbon atoms in their carboxyl moiety (Compounds 9, 10 and 26), Compound 9 was active at 42 mg/kg in the rat-MES model.

The insertion of a methylene spacer (Compound 26) between the amine and the phenylsulfonamide group of Compound 9 reduced its anticonvulsant activity in the rats-MES test (Table 1-3). The anticonvulsant potency varied among the analogous Compounds 11, 15, 16, 24, 27, 28 and 29, containing six carbon atoms in their carboxyl moiety (Table 3). Compound 11 was the most potent anticonvulsant in the rat-MES test followed by Compounds 16 and 29 (Table 1-3).

Insertion of one carbon atom spacer between the carboxamide and the aromatic sulfonamide moiety as in Compound 28 decreased the anticonvulsant activity. The rat-MES-ED₅₀ values were 9.9 mg/kg (Compound 11) and 24.7 mg/kg (Compound 28) and their respective PI value reduced from 51 (Compound 11) to 25 (Compound 28).

The widely used AED VPA is a branched (at carbon-2) monocarboxylic acid with eight carbons in its structure (1, Scheme 1). The 4-(valproylamido)-benzenesulfonamide (6, Scheme 2) showed remarkable anticonvulsant activity in mice-MES test. The anticonvulsant activity of additional six coupling products of 4-amino-benzenesulfonamide with branched short chain fatty acids, possessing eight carbon atoms in their carboxylic moiety (Compounds 12, 13, 18, 19, 23 and 25) was further studied. Among them Compound 23 was the most active in the rat-MES test (ED₅₀=16.7 mg/kg), following by Compound 25 and 18 (Table 3).

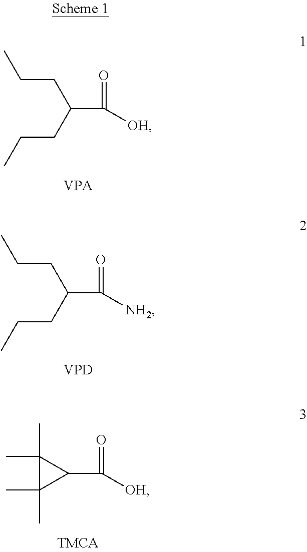

Structures of valproic acid (VPA, 1) and its corresponding amide derivatives: valpromide (VPD, 2) and VPA cyclopropyl analogue 2,2,3,3-tetramethylcyclopropane carboxylic acid (TMCA, 3)

Four 4-(carboxamido)-benzenesulfonamides (Compounds 14, 20, 21 and 22) possessing nine carbons in their carboxylic moiety were tested for anticonvulsant activity in rat-MES test (Table 1-3).

In the series of compounds presented in Table 2, Compounds 11, 16, 23 and 30 showed the highest anticonvulsant potency at the rat-MES seizure test (Table 3). 2-ethyl-N-(4-sulfamoyl-phenyl)-butyramide (Compound 11) was the most active anticonvulsant compound in these series and exhibited strong anticonvulsant properties (rat-MES test-ED₅₀=9.9 mg/kg, PI>51) and in mice it had an MES-ED₅₀ of 35 mg/kg (95% CI=29-39 mg/kg). However, in the scMet test it was inactive at doses up to 250 mg/kg.

Zonisamide, a widely used AED containing sulfonamide group in its structure, shown in Scheme 3, is less active than Compound 11 at the Rat-MES test (ED₅₀=21 mg/kg, PI=9.1). Zonisamide, as all synthesized compounds presented in Table 1, does not display anticonvulsant activity in the scMet test.

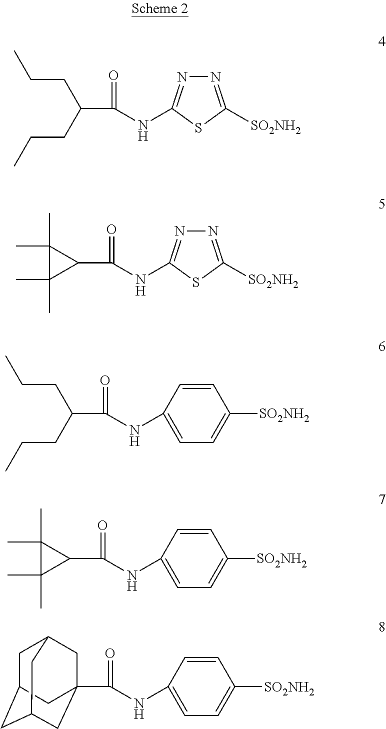

Structures of carboxamide derivatives containing 4-amino-benzenesulfonamide (6, 7, 8) and thiadiazolesulfonamide (4, 5)

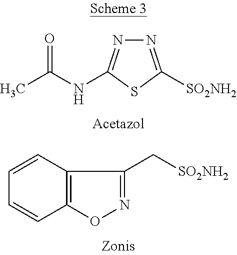

Clinically approved AEDs containing sulfonamide moieties.

Phenyl acetyl moiety was a lipophilic component, in a series of acetylurea derivatives. Phenylacetylurea emerged as the most potent anticonvulsant compound effective in grand mal and petit mal epilepsies as well as in psychomotor seizures. The synthesized derivative of phenyl acetic acid with 4-amino-benzenecarboxamide (Compound 30) were found to be more active than the N-(2-chloro-4-sulfamoyl-phenyl)-2-phenyl-acetamide in the rat-MES test (Table 3).

In a survey of 257-marketed CNS drugs, it was found that for potent drugs an optimal logP value (between 1 and 2) was required. For the compounds of the invention, the best anticonvulsant properties (Table 3) were found for Compounds 11, 16, 23 and 30, which were also highly lipophlic (Table 4), implying that penetration through the blood-brain bather is an important factor influencing the drugs efficacy. The correlation of the lipophilicity (ClogP) (Table 5) and in vivo anticonvulsant activity of the compounds presented in Table 1 was not found straightforward. The best anticonvulsant compound in this series (Compound 11) possessing ClogP of 1.54, whereas Compounds 13, 14, 20, 21 and 22 with higher ClogP value possessed lower anticonvulsant activities. It should be considered that the spatial arrangement in the chemical structure of the compounds in addition to ClogP values may also considerable influence on the anticonvulsant activity of the compounds in vivo.

TABLE 1

| | | Dose | MES^{a} | | scMet^{b} | | TOX^{c} | |
|---|---|---|---|---|---|---|---|---|
| Compd | Structure | (mg/kg) | 0.5h^{d} | 4h^{d} | 0.5h^{d} | 4h^{d} | 0.5h^{d} | 4h^{d} |
| 9 | 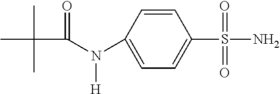 | 30 100 300 | 0/1 1/3 1/1 | 0/1 2/3 1/1 | 0/1 0/1 0/1 | 0/1 0/1 0/1 | 0/4 2/8 2/4 | 0/2 0/4 0/2 |
| 10 | 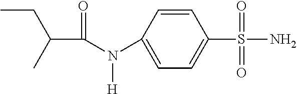 | 30 100 300 | 1/1 3/3 1/1 | 1/1 3/3 1/1 | 0/1 0/1 0/1 | 0/1 0/1 0/1 | 1/4 1/8 2/4 | 0/2 0/4 0/2 |
| 11 | 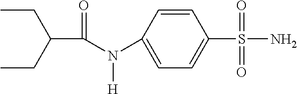 | 30 100 300 | 1/1 3/3 1/1 | 1/1 3/3 1/1 | 0/1 0/1 0/1 | 0/1 0/1 0/1 | 0/4 0/8 0/4 | 0/2 0/4 0/2 |
| 12 | 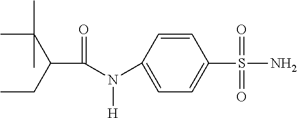 | 30 100 300 | 0/1 0/3 0/1 | 0/1 1/3 1/1 | 0/1 0/1 0/1 | 0/1 0/1 0/1 | 0/4 0/8 0/4 | 0/2 0/4 0/2 |
| 13 | 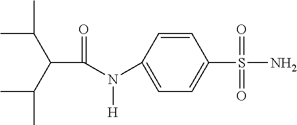 | 30 100 300 | 0/1 0/3 0/1 | 0/1 0/3 0/1 | 0/1 0/1 0/1 | 0/1 0/1 0/1 | 0/4 0/8 0/4 | 0/2 0/4 0/2 |
| 14 | 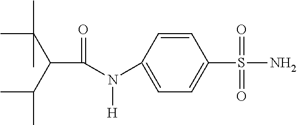 | 30 100 300 | 0/1 0/3 0/1 | 0/1 3/3 1/1 | 0/1 0/1 0/1 | 0/1 0/1 0/1 | 0/4 0/8 0/4 | 0/2 0/4 0/2 |
| 15 | 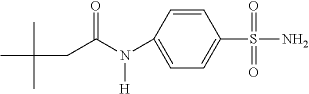 | 30 100 300 | 0/1 5/7 4/5 | 0/1 1/3 1/1 | 0/1 0/1 0/1 | 0/1 0/1 0/1 | 0/4 0/8 0/4 | 0/2 0/4 0/2 |
| 16 | 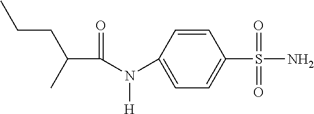 | 30 100 300 | 0/1 0/3 0/1 | 0/1 2/3 1/1 | 0/1 0/1 0/1 | 0/1 0/1 0/1 | 0/4 0/8 0/4 | 0/2 0/4 0/2 |
| 17 | 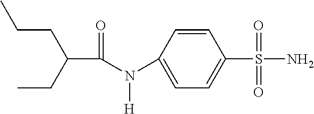 | 30 100 300 | 0/1 0/3 0/1 | 0/1 1/3 1/1 | 0/1 0/1 0/1 | 0/1 0/1 0/1 | 0/4 0/8 0/4 | 0/2 0/4 0/2 |
| 18 | 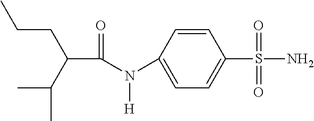 | 30 100 300 | 0/1 0/3 1/1 | 0/1 2/3 1/1 | 0/1 0/1 0/1 | 0/1 0/1 0/1 | 0/4 0/8 0/4 | 0/2 0/4 0/2 |
| 19 | 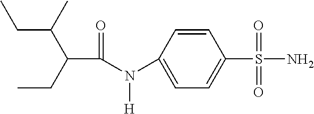 | 30 100 300 | 0/1 3/7 4/5 | 0/1 3/3 1/1 | 0/1 0/1 0/1 | 0/1 0/1 0/1 | 0/4 0/8 0/4 | 0/2 0/4 0/2 |
| 20 | 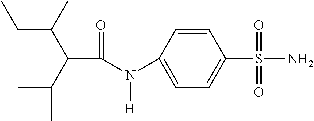 | 30 100 300 | 0/1 0/3 0/1 | 0/1 0/3 0/1 | 0/1 0/1 0/1 | 0/1 0/1 0/1 | 0/4 0/8 0/4 | 0/2 0/4 0/2 |
| 21 | 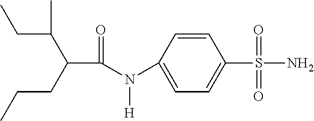 | 30 100 300 | 0/1 1/3 1/1 | 0/1 2/3 1/1 | 0/1 0/1 0/1 | 0/1 0/1 0/1 | 0/4 0/8 0/4 | 0/2 0/4 0/2 |
| 22 | 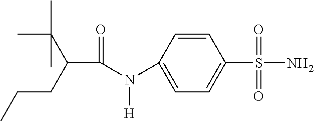 | 30 100 300 | 0/1 0/3 0/1 | 0/1 0/3 0/1 | 0/1 0/1 0/1 | 0/1 0/1 0/1 | 0/4 0/8 0/4 | 0/2 0/4 0/2 |
| 23 | 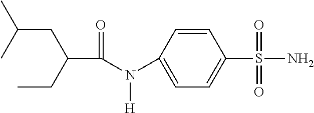 | 30 100 300 | 0/1 0/3 0/1 | 0/1 1/3 1/1 | 0/1 0/1 0/1 | 0/1 0/1 0/1 | 0/4 0/8 0/4 | 0/2 0/4 0/2 |
| 24 | 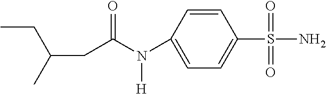 | 30 100 300 | 0/1 0/3 0/1 | 0/1 0/3 0/1 | 0/1 0/1 0/1 | 0/1 0/1 0/1 | 0/4 0/8 0/4 | 0/2 0/4 0/2 |
| 25 | 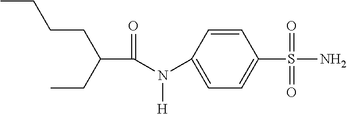 | 30 100 300 | 0/1 3/3 1/1 | 0/1 3/3 1/1 | 0/1 2/5 3/5 | 0/1 0/1 0/1 | 0/4 0/8 1/4 | 0/2 0/4 1/2 |
| 26 | 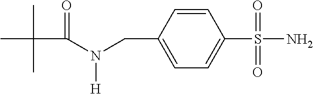 | 30 100 300 | 0/1 0/3 0/1 | 0/1 0/3 0/1 | 0/1 0/1 0/1 | 0/1 0/1 0/1 | 0/4 0/8 0/4 | 0/2 0/4 0/2 |
| 27 | 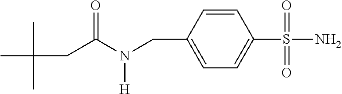 | 30 100 300 | 0/1 0/3 0/1 | 0/1 1/3 1/1 | 0/1 0/1 0/1 | 0/1 0/1 0/1 | 0/4 0/8 0/4 | 0/2 0/4 0/2 |
| 28 | 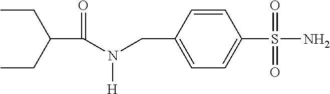 | 30 100 300 | 0/1 3/3 1/1 | 0/1 1/3 1/1 | 0/1 0/1 0/1 | 0/1 0/1 0/1 | 0/4 0/8 0/4 | 0/2 0/4 0/2 |
| 29 | 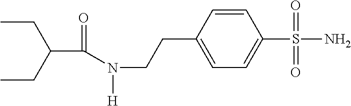 | 30 100 300 | 0/1 0/3 0/1 | 1/1 1/3 1/1 | 0/1 0/1 0/1 | 0/1 0/1 0/1 | 0/4 0/8 0/4 | 0/2 0/4 0/2 |
| 30 | 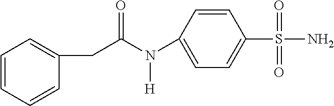 | 30 100 300 | 0/1 3/3 1/1 | 0/1 3/3 1/1 | 0/1 0/1 0/1 | 0/1 0/1 0/1 | 0/4 0/8 0/4 | 0/2 0/4 0/2 |

Anticonvulsant Activity and neurotoxicity of Compound 9-30, administered Intraperitoneally to Mice.
^{a}Maximal electroshock test (number of animals protected/number of animals tested).
^{b}Subcutaneous metrazol test (number of animals protected/number of animals tested).
^{c}Neurotoxicity (number of animals protected/number of animals tested).
^{d}Time after drug administration.

TABLE 2
Anticonvulsant [Anti-MES] Activity and neurotoxicity of Compounds
9-30 Administered Orally to Rats.

| | | Number of tested rats per time | | | | | |
|---|---|---|---|---|---|---|---|
| | Dose | after drug administered^{a} | | | | | |
| Compd | (mg/kg) | 15 min | 30 min | 1 h | 2 h | 4 h | TOX^{b} |
| 9 | 30 | 0/4 | 0/4 | 3/4 | 2/4 | 4/4 | — |
| 10 | 30 | 2/4 | 2/4 | 4/4 | 4/4 | 4/4 | — |
| 11 | 30 | 1/4 | 3/4 | 4/4 | 4/4 | 4/4 | — |
| 12 | 30 | | | | | | |
| 13 | 30 | 0/4 | 0/4 | 0/4 | 0/4 | 2/4 | — |
| 14 | 30 | | | | | | |
| 15 | 30 | 3/4 | 1/4 | 3/4 | 4/4 | 4/4 | |
| 16 | 30 | 1/4 | 1/4 | 4/4 | 4/4 | 4/4 | — |
| 17 | 30 | | | | | | |
| 18 | 30 | 0/4 | 0/4 | 4/4 | 3/4 | 2/4 | — |
| 19 | 30 | 0/4 | 0/4 | 3/4 | 4/4 | 3/4 | — |
| 20 | 30 | | | | | | |
| 21 | 30 | 0/4 | 1/4 | 1/4 | 2/4 | 1/4 | — |
| 22 | 30 | | | | | | |
| 23 | 30 | 2/4 | 1/4 | 3/4 | 3/4 | 3/4 | — |
| 24 | 30 | | | | | | |
| 25 | 30 | 0/4 | 0/4 | 4/4 | 3/4 | 4/4 | — |
| 26 | 30 | | | | | | — |
| 27 | 30 | 0/4 | 0/4 | 1/4 | 2/4 | 2/4 | — |
| 28 | 30 | 1/4 | 1/4 | 2/4 | 0/4 | 0/4 | — |
| 29 | 30 | 1/4 | 2/4 | 3/4 | 1/4 | 1/4 | — |
| 30 | 30 | 0/4 | 0/4 | 4/4 | 3/4 | 4/4 | — |

^{a}Number of animals protected/number of animals tested.
^{b}Neurotoxicity.

TABLE 3
Quantitative Anticonvulsant Data (Anti-MES
and Anti-scMet) in Rats Dosed Orally.

| | MES^{a} ED₅₀^{f} | | scMet^{c} ED₅₀^{f} | | TD₅₀^{e,f} |
|---|---|---|---|---|---|
| Compound | (mg/kg) | PI^{b} | (mg/kg) | PI^{d} | (mg/kg) |
| VPA | 485 (324-677) | 1.6 | 646 | 1.2 | 784 |
| | | | (466-869) | | (503-1176 |
| 9 | 42.2 (22.9-86.4) | >11.8 | — | — | >500 |
| 11 | 9.9 (6.5-14.5) | >50.5 | >250 | >2 | >500 |
| 16 | 10.8 (7.2-15.5) | >46.3 | >250 | >2 | >500 |
| 18 | 41.5 (23.9-63.4) | >12.0 | >250 | >2 | >500 |
| 23 | 16.7 (8.3-25.6) | >30.0 | — | — | >500 |
| 25 | 23.9 (16.3-32.6) | >21.0 | >250 | >2 | >500 |
| 28 | 24.7 (16.2-34) | >20.2 | >250 | >2 | >500 |
| 30 | 16.1 (8.5-24.8) | >31.0 | >250 | >2 | >500 |

^{a}Maximal electroshock test.
^{b}Protective index (PI = TD₅₀/ED₅₀) in the MES test.
^{c}Subcutaneous metrazol test.
^{d}Protective index (PI = TD₅₀/ED₅₀) in the scMet test.
^{e}Neurotoxicity.
^{f}The interval in parentheses stands for the 95% confidence interval.

TABLE 4
Lipophilicity Data (ClogP) of Compounds of the invention.

| Compound | ClogP |
|---|---|
| 9 | 0.883 |
| 10 | 1.013 |
| 11 | 1.542 |
| 12 | 2.34 |
| 13 | 2.34 |
| 14 | 2.739 |
| 15 | 1.502 |
| 16 | 1.542 |
| 17 | 2.071 |
| 18 | 2.47 |
| 19 | 2.47 |
| 20 | 2.869 |
| 21 | 2.999 |
| 22 | 2.869 |
| 23 | 2.47 |
| 24 | 1.632 |
| 25 | 2.6 |
| 26 | 0.35 |
| 27 | 0.969 |
| 28 | 1.009 |
| 29 | 1.078 |
| 30 | 1.184 |

Experimental Section:

All common reagents were obtained from Sigma-Aldrich and used without further purification. Acetone, dichloromethane (DCM), tetrahydrofuran (THF), petroleum ether and ethyl acetate are A.R. grade. Dry acetone, dichloromethane and tetrahydrofuran were obtained by reflux over CaH₂ for 2 h and distillation prior to use. N,N-dimethylpropyleneurea (DMPU) was obtained by refluxing over CaH₂ for 2 h and distillation at reduced pressure.

The reactions were followed by means of TLC analyses on aluminum sheets (Kieselgal 60 F₂₅₄, Merck) precoated silica gel on. ¹H NMR spectra were recorded on a Varian Mercury series NMR 300 spectrometer. Chemical shifts (δ scale) are reported in parts per million (ppm) relative to the indicated reference. Coupling constants (J) are given in (Hz).

Chemical structures of the newly synthesized compounds were assessed by ¹H NMR and elemental analysis. Melting point was determined on a 100-230 VAC MeI-temp capillary Melting point apparatus. Elemental analyses were preformed on a 2400-2 Perkin-Elmer C, H, N analyzer. C, H, N analyses of all newly synthesized compounds were within ±0.4 of theoretical values and thus were considered satisfactory.

The carboxylic acid used for the coupling with 4-amino-benzenesulfonamide derivatives were: 2,2-Dimethylpropionic acid (for the synthesis of compounds (9 and 26), valeric acid (for the synthesis of Compounds 16, 17, 18 and 22), isovelaric acid (for the synthesis of Compound 13), 3-methylvaleric acid (for the synthesis of Compounds 19, 20, 21 and 24), 4-methylvaleric acid (for the synthesis of Compound 23), 3,3-dimethylbutyric acid (for the synthesis of Compounds 12, 14, 15 and 27), butyric acid (for the synthesis of Compounds 10, 11, 28 and 29), hexanoic acid (for the synthesis of Compound 25) and phenylacetic acid (for the synthesis of Compound 30).

Some of the acids (e.g. 3-methylvaleric acid, 2,2-Dimethylpropionic acid, 3,3-Dimethylbutyric acid and phenyl acetic acid) were commercially available while others such as the branched carboxylic acids were prepared, as depicted in Scheme 5, by the conversion of the acid to the enolate by use of lithium diisopropylamine (LDA), followed by condensation with the appropriate alkyliodide to yield the corresponding branched carboxylic acid. The carboxylic acids were converted by thionyl chloride to the corresponding acylchloride and then coupled with 4-Amino-benzenesulfonamide or 4-aminomethyl-benzenesulfonamide or 4-aminoethyl-benzenesulfonamide in dry acetone and dry pyridine to yield Compounds 9-30. The synthesized products were purified by crystallization. ¹H NMR spectra of the synthesized compounds were measured in Dimethyl-sulfoxid-D₆ (DMSO). Elemental analyses were performed for all the synthesized compounds and were within ±0.4 of the theoretical values.

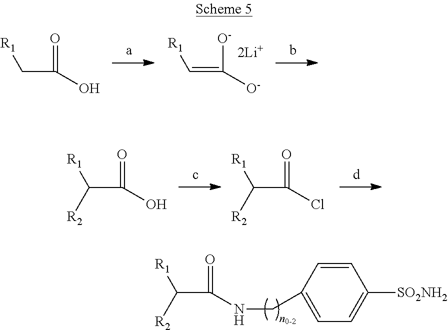

Synthesis of Compounds according to the present invention. ^{α}Reagents and conditions: (a) LDA, THF, −15° C., 20 min; (b) methyliodide or ethyliodide or propyliodide or iso-propyliodide, THF, 0° C., 12 hr; (c) SOCl₂, CH₂Cl₂, 25° C., 10 hr; (d) 4-Aminobenzene-sulfonamide or 4-aminomethylbenzenesulfonamide or 4-aminoethylbenzenesulfonamide, acetone, pyridine, room temperature, 12 hr.

### Example 1

### General Procedure for the Synthesis of Compounds 9-30

70 ml Anhydrous THF and 160 mmol diisopropylamine were added to a round-bottomed flask cooled to −15° C. under nitrogen (N₂) atmosphere, followed by a dropwise addition of 160 mmol n-butyllithium in order to prepare 160 mmol lithium diisopropylamine (LDA). The reaction mixture was stirred for 30 minutes and a 1:1 mixture of 10 ml dry THF and 72 mmol of either 2,2-Dimethylpropionic acid (for the synthesis of Compounds 9 and 26), valeric acid (for the synthesis of Compounds 16, 17, 18 and 22), isovelaric acid (for the synthesis of Compound 13), 3-methyl-valeric acid (for the synthesis of Compounds 19, 20, 21 and 24), 4-methylvaleric acid (for the synthesis of Compound 23), 3,3-dimethyl-butyric acid (for the synthesis of Compounds 12, 1.4, 15 and 27), butyric acid (for the synthesis of Compounds 10, 11, 28 and 29), hexanoic acid (for the synthesis of Compound 25), or phenylacetic acid (for the synthesis of Compound 30), was added and allowed to stir for additional 15 minutes below 0° C. 72 mmol DMPU was added dropwise after maintaining a temperature of 5° C., and allowed to stir for additional 30 minutes followed by a slow dropwise addition of a 1:1 solution containing 160 mmol of the corresponding alkyl iodide (either methyliodide, ethyliodide, propyliodide or isopropyliodide) in 10 ml anhydrous THF. The reaction mixture was allowed to stir at room temperature for 2 h. THF was distilled from the reaction mixture at 60-80° C. at normal pressure, and the oily product was dispersed in petroleum ether. 10% HCl solution was added until pH=1 was reached and the organic phase was separated from the aqueous phase and washed three times with brine. The aqueous phase was combined and extracted with petroleum ether (3×50 ml). The petroleum ether extracts were combined, dried over MgSO₄, filtered and evaporated to yield 89-97% oily products. The oily products were further distilled under reduced pressure to yield the pure corresponding acids.

The free carboxylic acids produced were chlorinated with thionyl chloride. The obtained acylchloride (9 mmol) was dissolved in 20 ml dry acetone and was added dropwise to stirred solution of suitable sulfonamide derivative (9.2 mmol) and pyridine (9.1=01) in 50 ml dry acetone. After addition, the reaction mixture was stirred for 12 h at room temperature. The organic solvent was then evaporated under vacuum and the residue dissolved in 100 ml ethyl acetate and washed three times with 20 ml of distilled water. 10% HCl solution was added until pH=1 was reached and the organic phase was separated from the aqueous phase and washed three times with brine. The aqueous phase was combined and extracted with ethyl acetate (3×50 ml). The ethyl acetate extracts were combined, dried over MgSO₄, filtered and evaporated.

The obtained products were purified by crystallization using ethanol/petroleum ether mixture (1:3).

### 2,2-Dimethyl-N-(4-sulfamoyl-phenyl)-propionamide (Compound 9)

White crystals; 88% yield; mp. 228-229° C.; ¹H NMR (300 MHz, CD₃SOCD₃, δ): 1.25 (s, 9H), 7.2 (s, 2H: SO₂NH₂), 7.6-7.8 (dd, J=9, 4H: H—Ar), 10.2 (s, 1H: NH). Anal. (C₁₁H₁₆N₂O₃S) C, H, N.

### 2-Methyl-N-(4-sulfamoyl-phenyl)-butyramide (Compound 10)

White crystals; 82% yield; mp. 205-207° C.; ¹H NMR (300 MHz, CD₃SOCD₃, δ): 0.8-0.9 (t, J=9, 3H), 1.1-1.15 (d, J=6.3, 3H), 1.31-1.46 (m, 1H), 1.5-1.65 (m, 1H), 2.4 (m, 1H), 7.2 (s, 2H: SO₂NH₂), 7.6-7.8 (m, 4H: H—Ar), 10.2 (s, 1H: NH). Anal. (C₁₁H₁₆N₂O₃S) C, H, N.

### 2-Ethyl-N-(4-sulfamoyl-phenyl)-butyramide (Compound 11)

White crystals; 63% yield; mp. 207-209° C.; ¹H NMR (300 MHz, CD₃SOCD₃, δ): 0.88-1.0 (t, J=9, 6H), 1.4-1.7 (br m, 4H), 2.2 (m, 1H), 7.2 (s, 2H: SO₂NH₂), 7.6-7.8 (m, 4H: H—Ar), 10.2 (s, 1H: NH). Anal. (C₁₂H₁₈N₂O₃S) C, H, N.

### 2-Ethyl-3,3-dimethyl-N-(4-sulfamoyl-phenyl)-butyramide (Compound 12)

White crystals; 81% yield; nip. 252-254° C.; ¹H NMR (300 MHz, CD₃SOCD₃, δ): 0.8 (t, J=7.2, 3H), 0.98 (s, 9H), 1.41-1.71 (br m, 2H), 2.1-2.18 (dd, J=97.5, 3, 1H), 7.2 (s, 2H: SO₂NH₂), 7.6-7.8 (m, 4H: H—Ar), 10.2 (s, 1H: NH). Anal. (C₁₄H₂₂N₂O₃S) C, H, N.

### 2-Isopropyl-3-methyl-N-(4-sulfamoyl-phenyl)-butyramide (Compound 13)

White crystals; 81% yield; mp. 244° C.; ¹H NMR (300 MHz, CD₃SOCD₃, δ): 0.85-0.95 (t, J=2.1, 12H), 1.8-2 (m, 3H), 7.2 (s, 2H: SO₂NH₂), 7.6-7.8 (m, 4H: H—Ar), 10.2 (s, 1H: NH). Anal. (C₁₄H₂₂N₂O₃S) C, H, N.

### 2-Isopropyl-3,3-dimethyl-N-(4-sulfamoyl-phenyl)-butyramide (Compound 14)

White crystals; 76% yield; mp. 248-250° C.; ¹H NMR (300 MHz, CD₃SOCD₃, δ): 0.9-1.1 (m, 12H), 1.1-1.3 (br m, 1H), 1.3-1.6 (br m, 1H), 1.8 (m, 2H), 2.2 (m, 1H), 7.2 (s, 2H: SO₂NH₂), 7.6-7.8 (m, 4H: H—Ar), 10.2 (s, 1H: NH). Anal. (C₁₅H₂₄N₂O₃S) C, H, N.

### 3,3-Dimethyl-N-(4-sulfamoyl-phenyl)-butyramide (Compound 15)

White crystals; 82% yield; nip. 211-213° C.; ¹H NMR (300 MHz, CD₃SOCD₃, δ): 1.0 (s, 9H), 2.2 (s, 2H), 7.2 (s, 2H: SO₂NH₂), 7.6-7.8 (m, 4H: H—Ar), 10.2 (s, 1H: NH). Anal. (C₁₂H₁₈N₂O₃S) C, H, N.

### 2-Methyl-N-(4-sulfamoyl-phenyl)-pentanamide (Compound 16)

White crystals; 93% yield; mp. 216-218° C.; ¹H NMR (300 MHz, CD₃SOCD₃, δ): 0.85 (t, J=7.2, 3H), 1.15 (d, J=6.9, 3H), 1.18-1.38 (m, 4H), 1.5-1.64 (m, 1H), 7.2 (s, 2H: SO₂NH₂), 7.6-7.8 (m, 4H: H—Ar), 10.2 (s, 1H: NH). Anal. (C₁₂H₁₈N₂O₃S) C, H, N.

### 2-Ethyl-N-(4-sulfamoyl-phenyl)-pentanamide (Compound 17)

White solid; 83% yield; mp. 203-205° C.; ¹H NMR (300 MHz, CD₃SOCD₃, δ): 0.91 (t, J=7, 6H), 1.19-1.6 (br m, 6H), 2.2-2.4 (m, 1H), 7.2 (s, 2H: SO₂NH₂), 7.6-7.8 (m, 4H: H—Ar), 10.2 (s, 1H: NH). Anal. (C₁₃H₂₀N₂O₃S) C, H, N.

### 2-Isopropyl-N-(4-sulfamoyl-phenyl)-pentanamide (Compound 18)

White crystals; 89% yield; mp. 217-219° C.; ¹H NMR (300 MHz, CD₃SOCD₃, δ): 0.78-0.9 (m, 9H), 1.1-1.23 (m, 2H), 1.3-1.4 (bm, 2H), 1.65-1.8 (m, 1H), 2.05-2.2 (m, 1H), 7.2 (s, 2H: SO₂NH₂), 7.6-7.8 (m, 4H: H—Ar), 10.2 (s, 1H: NH). Anal. (C₁₄H₂₂N₂O₃S) C, H, N.

### 2-Ethyl-3-methyl-N-(4-sulfamoyl-phenyl)-pentanamide (Compound 19)

White crystals; 71% yield; mp. 203-205° C.; ¹H NMR (300 MHz, CD₃SOCD₃, δ): 0.7-0.9 (m, 9H), 1.0-1.2 (m, 1H), 1.4-1.68 (m, 4H), 2.08-2.22 (m, 1H), 7.2 (s, 2H: SO₂NH₂), 7.6-7.8 (m, 4H: H—Ar), 10.2 (s, 1H: NH). Anal. (C₁₄H₂₂N₂O₃S) C, H, N.

### 2-isopropyl-3-methyl-N-(4-sulfamoyl-phenyl)-pentanamide (Compound 20)

White crystals; 69% yield; mp. 230-233° C.; ¹H NMR (300 MHz, CD₃SOCD₃, 6): 0.8-1.0 (m, 12H), 1.0-1.2 (m, 1H), 1.4-1.58 (m, 1H), 1.7-1.8 (m, 1H), 1.9-2.08 (m, 1H), 2.08-2.2 (m, 1H), 7.2 (s, 2H: SO₂NH₂), 7.6-7.8 (m, 4H: H—Ar), 10.2 (s, 1H: NH). Anal. (C₁₄H₂₂N₂O₃S) C, H, N.

### 2-propyl-3-Methyl-N-(4-sulfamoyl-phenyl)-pentanamide (Compound 21)

White crystals; 72% yield; mp. 223-226° C.; ¹H NMR (300 MHz, CD₃SOCD₃, δ): 0.8-0.98 (m, 9H), 1.05-1.3 (br m, 2H), 1.3-1.63 (br m, 5H), 2.25 (m, 1H), 7.2 (s, 2H: SO₂NH₂), 7.6-7.8 (m, 4H: H—Ar), 10.2 (s, 1H: NH). Anal. (C₁₅H₂₄N₂O₃S) C, H, N.

### 2-tert-Butyl-N-(4-sulfamoyl-phenyl)-pentanamide (Compound 22)

White crystals; 60% yield; mp. 260-264° C.; ¹H NMR (300 MHz, CD₃SOCD₃, δ): 0.82-0.89 (t, 3H), 0.89-1.0 (s, 9H), 1.08-1.22 (m, 1H), 1.3-1.44 (m, 2H), 1.56-1.78 (m, 1H), 2.2 (dd, J=12, 1H), 7.2 (s, 2H: SO₂NH₂), 7.6-7.8 (m, 4H: H—Ar), 10.2 (s, 1H: NH). Anal. (C₁₅H₂₄N₂O₃S) C, H, N.

### 2-Ethyl-4-methyl-N-(4-sulfamoyl-phenyl)-pentanamide (Compound 23)

White crystals; 87% yield; mp. 199-200° C.; ¹H NMR (300 MHz, CD₃SOCD₃, 6): 0.85-1.0 (m, 9H), 1.2-1.38 (m, 1H), 1.4-1.65 (br m, 4H), 2.3-2.5 (m, 1H), 7.2 (s, 2H: SO₂NH₂), 7.6-7.8 (m, 4H: H—Ar), 10.2 (s, 1H: NH). Anal. (C₁₄H₂₂N₂O₃S) C, H, N.

### 3-Methyl-N-(4-sulfamoyl-phenyl)-pentanamide (Compound 24)

White crystals; 76% yield; mp. 204-206° C.; ¹H NMR (300 MHz, CD₃SOCD₃, δ): 0.8-0.92 (m, 6H), 1.1-1.23 (m, 1H), 1.23-1.4 (m, 1H), 1.8-1.98 (m, 1H), 2.09-2.2 (dd, J=3, J=9, 1H), 2.3-2.34 (dd, J=3, J=9, 1H), 7.2 (s, 2H: SO₂NH₂), 7.6-7.8 (m, 4H: H—Ar), 10.2 (s, 1H: NH). Anal. (C₁₂H₁₈N₂O₃S) C, H, N.

### 2-Ethyl-N-(4-sulfamoyl-phenyl)-hexanamide (Compound 25)

White crystals; 90% yield; mp. 188-190° C.; ¹H NMR (300 MHz, CD₃SOCD₃, δ): 0.78-0.86 (m, 6H), 1.1-4.3 (m. 4H), 1.3-1.65 (bin, 4H), 2.2-2.4 (m, 1H), 7.2 (s, 2H: SO₂NH₂), 7.6-7.8 (m, 4H: H—Ar), 10.2 (s, 1H: NH). Anal. (C₁₄H₂₂N₂O₃S) C, H, N.

### 2,2-Dimethyl-N-(4-sulfamoyl-benzyl)-propionamide (Compound 26)

White crystals; 70% yield; mp. 157-158° C.; ¹H NMR (300 MHz, CD₃SOCD₃, δ): 1.15 (s, 9H), 4.32 (d, J=7.5, 2H), 7.3 (s, 2H: SO₂NH₂), 7.4 (d, J=12, 2H: H—Ar), 7.8 (d, J=12, 2H—Ar), 10.2 (s, 1H: NH). Anal. (C₁₂H₁₈N₂O₃S) C, H, N.

### 3,3-Dimethyl-N-(4-sulfamoyl-benzyl)-butyramide (Compound 27)

White crystals; 80% yield; mp. 205-207° C.; ¹H NMR (300 MHz, CD₃SOCD₃, δ): 0.98 (s, 9H), 2.0 (s, 2H), 4.3 (d, J=6, 2H), 7.3 (s, 2H: SO₂NH₂), 7.4 (d, J=12, 2H: H—Ar), 7.8 (d, J=12, 2H: H—Ar), 10.2 (s, 1H: NH). Anal. (C₁₃H₂₀N₂O₃S) C, H, N.

### 2-Ethyl-N-(4-sulfamoyl-benzyl)-butyramide (Compound 28)

White crystals; 62% yield; mp. 148-150° C.; ¹H NMR (300 MHz, CD₃SOCD₃, δ): 0.88-1.0 (t, J=9, 61-1), 1.4-1.7 (br m, 4H), 2.2 (m, 1H); 4.38 (d, J=6, 2H), 7.2 (s, 2H: SO₂NH₂), 7.4 (d, J=12, 2H: H—Ar), 7.8 (d, J=12, 2H: H—Ar), 10.2 (s, 1H: NH). Anal. (C₁₃H₂₀N₂O₃S) C, H, N.

### 2-Ethyl-N-[2-(4-sulfamoyl-phenyl)-ethyl]-butyramide (Compound 29)

White crystals; 35% yield; mp. 181-183° C.; ¹H NMR (300 MHz, CD₃SOCD₃, δ): 0.7-0.8 (t, J=9, 6H), 1.2-1.5 (br m, 4H), 1.8-2.0 (m, 1H), 2.8 (t, J=9, 2H), 3.35 (q, J=6, 2H), 7.2 (s, 2H: SO₂NH₂), 7.4 (d, J=12, 2H: H—Ar), 7.8 (d, J=12, 2H: H—Ar), 10.2 (s, 1H: NH). Anal. (C₁₄H₂₂N₂O₃S) C, H, N.

### 2-Phenyl-N-(4-sulfamoyl-phenyl)-acetamide (Compound 30)

White crystals; 85% yield; mp. 210-211° C.; ¹H NMR (300 MHz, CD₃SOCD₃, δ): 3.63 (s, 2H), 7.2 (s, 2H: SO₂NH₂), 7.3 (m, 4H; H—Ar), 7.6-7.8 (m, 4H: H—Ar), 10.2 (s, 1H: NH). Anal. (C₁₄H₁₄N₂O₃S) C, H, N.

### Example 2

### Biological Testing

The evaluation of the anticonvulsant activity in the maximal electroshock seizure test (MES) and subcutaneous metrazol seizure threshold test (scMet) and the determination of neurotoxicity in the rotorod test, positional sense test, and others were performed according to the protocols described in White, H. S., Woodhead J. H., Wilcox K. S., Stables J. P., Kupferberg H. J., Wolf H. H., Discovery and Preclinical Development of Antiepileptic Drugs; 5^{th} ed.; Lippincott Williams &Wilkins: New york, 2002; 36-48.

### Example 3

### Preparation of Compounds for Testing

The tested compounds were suspended in 0.5% methylcellulose and administered (a) intraperitioneally (ip) to adult male CF no. 1 albino mice (18-25 g) in volume of 0.01 mL/g body weight and (b) orally to adult male Sprague-Dawley albino rats (100-150 g) in volume of 0.04 mL per 10 g of body weight. The pentylenetetrazol solution at convulsing dose was prepared by sufficient dissolution of pentylenetetrazol in 0.9% saline to make 0.85% solution for administration to mice and a 2.82% solution for administration to rats.

### Example 4

### Determination of the Median Effective Dose (ED₅₀) and the Median Neurotoxic Dose (TD₅₀)

For the determination of the ED₅₀ by the respective anticonvulsant procedure, doses of the tested compounds were varied until a minimum of three to four points are established between the dose level of 0% protection and of 100% protection. These data were subjected to the FORTRAN probit analysis program and the ED₅₀ and 95% confidence intervals were calculated. The TD₅₀ was determined by varying the dose of the tested compounds until four points were established between the dose level that induced no signs of minimal motor impairment in any of the animals and the dose at which all the animals were considered impaired. The TD₅₀ and the 95% confidence intervals were calculated by FORTRAN probit analysis. The PIs were calculated by dividing the TD₅₀ by the ED₅₀.

### Example 5

### Calculation of C log P

C log P was calculated by means of ChemDraw-Ultra Software 8.

1-50. (canceled)

51. A compound of the general formula (I): 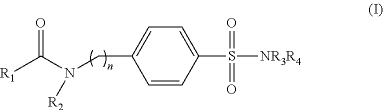
wherein
R₁ is selected from the group consisting of C₄-C₉ alkyl and C₆-C₁₀ alkylenearyl;
R₂ is —H;
each of R₃ and R₄ is —H; and
n is 0;
wherein said C₄-C₉-alkyl is selected from the group consisting of sec-butyl, 2-pentyl, 3-pentyl, 3-hexyl, 1-iso-propylbutyl, 3-heptyl, 2,2,4-trimethyl-pent-3-yl, 2,2-dimethyl-prop-1-yl, 3-methyl-hept-4-yl, 5-methyl-hex-3-yl and 2-methyl-but-1-yl.

52. The compound according to claim 51, wherein R₁ is 3-pentyl.

53. A compound selected from the group consisting of Compounds 10, 11, 14, 15, 16, 17, 18, 21, 23, 24, and 25 of Table 1.

54. A method of treating or preventing a diseases or disorder in a subject, said method comprising administering to said subject a pharmaceutical composition comprising a compound according to claim 51.

55. The method according to claim 54, wherein said disease or disorder is epilepsy.

56. A method for the treatment and/or prevention of epilepsy, said method comprising administering to a subject in need thereof a pharmaceutical composition comprising a compound of formula (I): 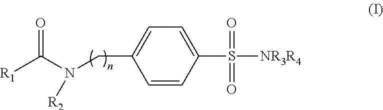
wherein:
R₁ is selected from the group consisting of C₄-C₉ alkyl, and C₆-C₁₀ alkylenearyl;
R₂ is —H;
n is 0 or 1; and
each of R₃ and R₄ is —H,
wherein said C₄-C₉-alkyl is selected from the group consisting of sec-butyl, tert-butyl, 2-pentyl, 3-pentyl, 3-hexyl, 1-iso-propylbutyl, 3-heptyl, 2,2,4-trimethyl-pent-3-yl, 2,2-dimethyl-prop-1-yl, 3-methyl-hept-4-yl, 5-methyl-hex-3-yl and 2-methyl-but-1-yl.

57. The method according to claim 56, wherein said compound is selected from the group consisting of Compounds 9, 10, 11, 14, 15, 16, 17, 18, 21, 23, 24, 25 and 28 of Table 1.

58. The method according to claim 56, wherein R₁ is a C₆-C₁₀ alkylenearyl.

59. The method according to claim 58, wherein said C₆-C₁₀ alkylenearyl is selected from the group consisting of —CH₂—Ar, —CH₂—CH₂—Ar and —CH₂CH₂CH₂—Ar.

60. The method according to claim 59, wherein said aryl group (—Ar) is phenyl.

61. A method for treating or preventing a disease or disorder, said method comprising administering to a subject in need thereof a pharmaceutical composition comprising a compound of the formula (VII): 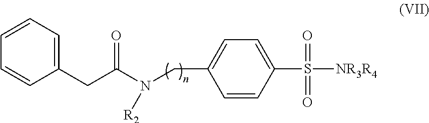
wherein
R₁ is selected from the group consisting of optionally substituted C₄-C₉ alkyl, optionally substituted C₆-C₁₀ aryl, optionally substituted C₆-C₁₀ alkylenearyl and optionally substituted C₅-C₁₀-heteroaryl;
R₂ is selected from the group consisting of —H and optionally substituted C₁-C₆ alkyl;
n is 0, 1, 2, 3 or 4; and
each of R₃ and R₄, independently of each other, is selected from the group consisting of —H, optionally substituted C₁-C₆ alkyl, optionally substituted C₆-C₁₀ aryl and optionally substituted C₅-C₁₀-heteroaryl.

62. The method according to claim 61, wherein R₂ is —H.

63. The method according to claim 61, wherein each of R₃ and R₄ is —H.

64. The method according to claim 61, wherein each of R₂, R₃ and R₄ is —H.

65. The method according to claim 61, wherein said compound is Compound 30 of Table 1.

66. A composition comprising at least one compound according claim 61.

67. The composition according to claim 66, being a pharmaceutical composition.

68. The composition according to claim 67, for use in the treatment or prevention of epilepsy.

69. The composition according to claim 66, comprising at least one compound selected from the group consisting of Compounds 9, 10, 11, 14, 15, 16, 17, 18, 21, 23, 24, 25, 28 and 30 of Table 1.
